(19) United States
(12) Patent Application Publication
(10) Pub. No.: US 2005/0089874 A1
(43) Pub. Date: Apr. 28, 2005

(54) Amplification of signal using a bead-based oligonucleotide assay

(76) Inventors: Suzanne M. Torontali, Cincinnati, OH (US); Mary Lynn Jump, Fairfield, OH (US); Kenton D. Juhlin, Milford, OH (US); Brian D. Richardson, Cincinnati, OH (US); Jay P. Tiesman, Hamilton, OH (US); Jorge M. Naciff, Cincinnati, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/693,025

(22) Filed: Oct. 24, 2003

(51) Int. Cl. C12Q 1/68; G06F 19/00; G01N 33/48; G01N 33/50

(52) U.S. Cl. 435/6; 702/20

(57) ABSTRACT

The present invention regards amplification of a signal from a hybridization-based oligonucleotide assay. In some embodiments, a bead comprises an oligonucleotide hybridized to a labeled polynucleotide from a sample, and a signal generated from a complex thereof is amplified through labeled antibodies directed to a receptor for the label. In particular embodiments, the assay provides information on gene expression.

### FIELD OF THE INVENTION

The present invention is directed to the fields of molecular biology, sequence analysis and gene expression analysis. More specifically, the field of the invention regards amplifying a signal from a bead-based oligonucleotide gene expression assay.

### BACKGROUND OF THE INVENTION

A variety of applications, including gene expression profiling, sequencing of polynucleotides, detection of genetic mutations, genotyping, species identification and phenotypic characterization, exposure to specific chemicals (toxic and/or therapeutic), and the like utilize nucleic acid sequence detection methods. Methods for the detection of nucleic acid sequences have suffered from drawbacks including background noise, time and labor requirements, lack of specificity, and lack of sensitivity. Some detection methods utilize arrays of polymers, such as nucleic acids that may be screened for specific binding to a target, such as a complementary nucleotide. Gene expression studies have been accelerated recently by the use of microarrays. By assaying the expression of thousands of genes at a time, microarrays have led to the discovery of dozens of genes involved in particular biochemical processes. The next step in these studies focuses on a subset of significant genes identified using the arrays.

McHugh et al. (1988) concerns microspheres comprising viral antigens subjected to human antibodies that were detected using biotinylated antihuman IgG, followed by streptavidin-PE.

Lindmo et al. (1990) regards an assay utilizing two particle types having the same specificity but different affinity for a secondary biotin-streptavidin-phycoerythrin-conjugated antibody directed against a carcinoembryonic antigen epitope.

Spycher et al. (1991) is directed to microspheres exposed to human serum followed by biotinylated monoclonal anti-C3d or anti-C4d antibody, and phycoerythrin-streptavidin, wherein the fluorescence was measured by flow cytometry and corresponded to the amount of deposited C3 and C4.

Bhalgat et al. (1998) concerns microspheres having one of two different fluorophores, wherein the microspheres were conjugated to streptavidin for selecting cell surface markers labeled with a biotinylated primary.

Dunbar et al. (2003) describes LabMAP® microspheres for detecting bacterial pathogens, wherein microspheres coupled to a capture antibody specific for a particular microorganism were subjected to samples comprising microorganism-specific antigens, which were then subjected to biotinylated detection antibodies and strepavidin-R-phycoerythrin.

Yang et al. (2001) and U.S. patent application No. 2002/0034753 are directed to the status of providing microspheres linked to a capture probe that has sequence complementary to a first segment of a sequence of a single-stranded target nucleic acid; contacting the substrate with a nucleic acid sample that hybridizes to the capture probe, wherein upon the hybridization at least a second segment of the sequence of the target nucleic acid remains single stranded; exposing the substrate to conditions for complementing at least a second segment of the target nucleic acid, wherein the complementing nucleic acid comprises nucleotides having a label capable of enhancing sensitivity of detection of the complementing nucleic acid; and analyzing the label to determine presence or absence of the target nucleic acid in the nucleic acid sample.

U.S. Pat. No. 6,203,989 and U.S. patent application No. 2001/0041335 regard methods and compositions for amplifying signals in specific binding assays, such as by hybridizing a nucleic acid target to a nucleic acid probe, wherein the target comprises a binding ligand, contacting the hydridized target with a receptor comprising multiple sites capable of binding the binding ligand to complex the receptor to the binding ligand, contacting the receptor with a reagent, comprising a plurality of the binding ligands, to complex the reagent to the receptor; and detecting the presence of the complexed reagent. In particular embodiments, FIG. 1 illustrates the nucleic acid probes as being immobilized on a linear solid substrate.

### SUMMARY OF THE INVENTION

It is an object of the invention to provide materials for the detection of polymers, particularly nucleic acids. It is a particular object of the invention to provide methods and compounds for amplifying labeling signals used in the detection of nucleic acid sequences in specific binding assays. It is a further object of the invention to provide methods and compounds that permit nucleic acid sequences to be detected specifically and rapidly with high sensitivity and high resolution.

The present invention regards a high-throughput gene expression assay to evaluate particular gene expression scenarios. Several improvements on existing bead-based assays that are highly correlated in signal and gene regulation to microarray technologies are provided in the present invention. These improvements include at least the exemplary streptavidin phycoerythrin amplification utilizing biotinylated anti-streptavidin in addition to optimization of time, temperature, and other assay conditions. Using this methodology, detection levels down to 1 attomole have been achieved, detecting rare messages in complex cRNA samples, for example, using as little as 1.0 μg. This assay offers increased throughput with decreased costs compared to existing microarray technologies. In particular embodiments, the amplification technique is applied to protein and/or gene expression assays, such as with total RNA.

In specific embodiments, the invention utilizes assays based, for example, on commercially available oligonucleotide hybridization systems, such as the Luminex® xMAP® system. This system is a rapid multiplexed assay platform that quantifies up to 100 distinct analytes simultaneously in a single sample in a 96-well plate format. The xMAP® system is based on polystyrene microspheres, internally dyed with different ratios of two spectrally distinct fluorochromes that provide a spectral array of 100 distinct elements. Using the xMAP® system, the present inventors developed an expression profiling assay specific for a particular number of different genes of interest using beads coupled with optimally selected oligonucleotides. This assay would also apply to a full set of 100 analytes, as referred to above.

In an embodiment of the present invention, there is a method for amplifying a signal for detection of a polynucleotide, comprising the steps of (a) providing at least one microsphere linked to at least one pre-optimized oligonucleotide; (b) hybridizing a labeled target polynucleotide to the oligonucleotide to form an oligonucleotide/target polynucleotide complex, wherein the complex comprises a detectable signal through the binding of a receptor to the label; and (c) providing a labeled ligand for the receptor, wherein when the ligand binds the receptor, the signal is amplified. In specific embodimetns, the pre-optimized oligonucleotide is selected with an algorithm.

An algorithm for selecting a pre-optimized oligonucleotide may utilize at least one of the following selection criteria: (a) selecting at least one perfect match pre-optimized oligonucleotide, wherein the selected at least one perfect match pre-optimized oligonucleotide has an acceptable measure of correlation with a standard gene expression value; (b) selecting at least one perfect match and minus mismatch pre-optimized oligonucleotide pair, wherein within a pair the selected at least one perfect match pre-optimized oligonucleotide minus the mismatch pre-optimized oligonucleotide has an acceptable measure of correlation with a standard gene expression value; (c) selecting at least one pair of pre-optimized oligonucleotides from different pre-optimized oligonucleotide sets, wherein the ratio of signals in the pre-optimized oligonucleotides in the at least one pair of pre-optimized oligonucleotides has an acceptable correlation with a standard signal ratio; and (d) selecting at least one perfect match pre-optimized oligonucleotide, wherein the perfect match pre-optimized oligonucleotide has an acceptable relative standard deviation.

In specific embodiments, the pre-optimized oligonucleotide is further defined as being selected by the steps of: providing a sample comprising at least one target polynucleotide; subjecting the sample to an array of oligonucleotides, wherein the hybridization of the target polynucleotide to at least one oligonucleotide in the array provides a detectable hybridization fingerprint; and identifying at least one optimal oligonucleotide from the fingerprint. The pre-optimized oligonucleotide may be further defined as being selected by the steps of: providing a sample comprising a plurality of target polynucleotides, said target polynucleotides defined as RNA polynucleotides from more than one gene; subjecting said sample to an array of oligonucleotides, wherein the hybridization of more than one different RNA polynucleotide to a respective oligonucleotide in the array provides a detectable hybridization fingerprint for more than one gene; and identifying at least one optimal oligonucleotide for said more than one gene from said fingerprint. In a specific embodiment of the present invention, the identifying step utilizes an algorithm to identify the oligonucleotide.

In other specific embodiments of the present invention, the algorithm identifies an oligonucleotide having complete complementarity to at least a portion of a target polynucleotide. The target polynucleotide may be comprised in a plurality of RNA polynucleotides, and the concentration of the plurality may be from about 1 μg to about 10 μg.

In additional specific embodiments, the ligand comprises an antibody. Also, the label of the target polynucleotide and/or the label of the ligand may comprise a fluorescent label, an enzyme label, and/or a gold label. In some embodiments, the label of the target polynucleotide and the label of the ligand are substantially similar or identical.

In further specific embodiments, the microsphere is comprised in a plurality of microspheres and the target polynucleotide is comprised in a plurality of RNA polynucleotides. The plurality of RNA polynucleotides may be comprised in a mRNA-containing sample, and the method may be further defined as a method for providing mRNA expression profiling information. In specific embodiments, at least one microsphere in the plurality of microspheres comprises different oligonucleotides from the oligonucleotides of another microsphere in the plurality. At least one microsphere in the plurality may comprise more than one non-identical pre-optimized oligonucleotide having sequence complementary to the same RNA polynucleotide.

In another embodiment of the present invention, there is a composition, comprising: a plurality of microspheres, each microsphere linked to at least one pre-optimized oligonucleotide, wherein the oligonucleotide is hybridized to a labeled RNA polynucleotide forming an oligonucleotide/labeled RNA polynucleotide hybridized complex, and wherein the complex comprises a detectable signal through the binding of a receptor to the label, the signal amplified upon binding of a labeled ligand for the receptor. At least one microsphere in the plurality of microspheres may comprise different oligonucleotides from the oligonucleotides of another microsphere in said plurality. Also, at least one microsphere in the plurality may comprise more than one non-identical pre-optimized oligonucleotide each having sequence complementary to the same RNA polynucleotide.

In an additional embodiment of the present invention, there is a method of optimizing an oligonucleotide hybridization-based assay, comprising the steps of: providing a sample comprising at least one target polynucleotide; subjecting the sample to an array of oligonucleotides, wherein the hybridization of the target polynucleotide to at least one oligonucleotide in the array provides a detectable hybridization fingerprint; identifying at least one optimal oligonucleotide from the fingerprint, wherein the identifying step utilizes an algorithm defined by at least one of the following selection criteria: (a) selecting at least one perfect match pre-optimized oligonucleotide, wherein the selected at least one perfect match pre-optimized oligonucleotide has an acceptable measure of correlation with a standard gene expression value; (b) selecting at least one perfect match and minus mismatch pre-optimized oligonucleotide pair, wherein within a pair the selected at least one perfect match pre-optimized oligonucleotide minus the mismatch pre-optimized oligonucleotide has an acceptable measure of correlation with a standard gene expression value; (c) selecting at least one pair of pre-optimized oligonucleotides from different pre-optimized oligonucleotide sets, wherein the ratio of signals in the pre-optimized oligonucleotides in the at least one pair of pre-optimized oligonucleotides has an acceptable correlation with a standard signal ratio; and (d) selecting at least one perfect match pre-optimized oligonucleotide, wherein the perfect match pre-optimized oligonucleotide has an acceptable relative standard deviation; and subjecting the optimal oligonucleotide to an oligonucleotide hybridization-based assay.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description.

### DETAILED DESCRIPTION OF THE INVENTION

Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

As used herein, the term “fingerprint” refers to a signature pattern of hybridization of at least one target polynucleotide in a particular sample with one or more oligonucleotide probes, such as immobilized oligonucleotide probes. In a specific embodiment, the fingerprint provides information for at least one hybridization pattern for a plurality of different target polynucleotides at least some of which comprise sequence from different genes (or their representative mRNAs or cRNAs).

As used herein, the term “hybridization” refers to the association between two nucleic acids, for example the non-covalent interaction through base pair hydrogen bonding and base stacking.

As used herein, the term “microsphere” refers to a spherical structure, such as a generally spherical structure, that comprises a detectable signature signal on and/or in the structure, for example through at least one identifiable label. In specific embodiments, the microsphere comprises at least one oligonucleotide, such as attached to thereon. In a specific embodiment, the microsphere may be referred to as a bead. A particular microsphere in a plurality of microspheres may be distinguishable from another by at least one characteristic. For example, microspheres may be distinguished based on at least one label, such as a calorimetric or fluorescent label on and/or in the microsphere; based on size; charge; and so forth.

Polynucleotides, including oligonucleotides, may be utilized in the present invention. A skilled artisan recognizes that the term “polynucleotide” or “nucleic acid” as used herein refers to a polymeric form of nucleotides of any length, either ribonucleotides or deoxyribonucleotides, that comprise purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. A polynucleotide may comprise modified nucleotides, such as methylated nucleotides and nucleotide analogs, and in specific embodiments, the polynucleotides are labeled, such as by having been generated through polymerization in the presence of labeled nucleotides.

As used herein, “stringency” refers to the conditions of a particular hybridization reaction that affect the extent to which nucleic acids hybridize. The stringency of the hybridization conditions can be chosen so that nucleic acid duplexes may be selected based on their degree of complementarity. For example, high stringency is associated with a lower probability for the formation of a duplex containing mismatched bases, and, therefore, the higher the stringency, the greater the probability that two single-stranded nucleic acids having a corresponding mismatched duplex will remain unhybridized. Generally, conditions that increase stringency, thereby selecting for the formation of greater complementarity between hybridized molecules, include higher temperature, lower ionic strength, and/or presence or absence of solvents. Alternatively, at lower stringency the probability of formation of a mismatched duplex is increased. Lower stringency is favored by lower temperature, higher ionic strength, and/or lower or higher concentrations of solvents (such as reduced concentrations of formamide or dimethyl sulfoxide). The duration of the hybridization reaction and the concentration of reactants (i.e., single stranded nucleic acid) can also affect stringency, with short reaction times and low reactant concentrations favoring higher stringency. A skilled artisan recognizes that the appropriate stringency that will permit selection of a perfectly-matched duplex, as opposed to a duplex containing one or more mismatches, may generally be determined empirically. Means for adjusting the stringency of a hybridization reaction are well-known to those of skill in the art. Nucleic acid hybridization assay procedures and conditions developed in the art may be used in the invention, as described, for example in: Maniatis et al., “Molecular Cloning: A Laboratory Manual” 2nd Ed., Cold Spring Harbor, N.Y., 1989; Berger and Kimmel, “Methods in Enzymology,” Vol. 152, “Guide to Molecular Cloning Techniques”, Academic Press, Inc., San Diego, Calif., 1987; Young and Davis, Proc. Natl. Acad. Sci., U.S.A., 80:1194 (1983).

As used herein, the term “target polynucleotide” refers to at least one polynucleotide being tested for the ability to hybridize to one or more immobilized oligonucleotide(s) on a microsphere of the present invention. In a specific embodiment, the target polynucleotide is labeled, such as with biotin. The target polynucleotide may be labeled at the 5′ end and/or the 3′ end, and/or it may be labeled at one or more internal nucleotides. In other specific embodiments, the target polynucleotide is comprised within a plurality of polynucleotides, which may be other target polynucleotides having different sequences. The target polynucleotide may be any kind of nucleic acid, but in particular embodiments it is an RNA polynucleotide, and in further particular embodiments it is an mRNA or cRNA polynucleotide. In additional embodiments, the target polynucleotide is comprised within a sample.

The Present Invention

The methods and compositions disclosed herein may be used in a variety of applications related to assaying for hybridization of a target polynucleotide to an oligonucleotide probe and amplification of a signal generated thereby. In one embodiment, one or more target polynucleotides comprising different target sequences are screened for hybridization to a high density array of immobilized oligonucleotide probes comprising different sequences, and an amplified signal is detected.

Methods and compounds are provided for signal amplification in the detection of at least one target molecule by utilizing specific binding assays. Although exemplary oligonucleotides and RNA polynucleotides are provided in detail herein, the methods and compounds disclosed herein may also be used to detect the binding of other molecules, such as polypeptides.

In one embodiment, methods are provided for detecting a nucleic acid target, wherein the method comprises hybridizing a nucleic acid target (such as an RNA polynucleotide), preferably labeled, to an immobilized oligonucleotide comprised on a microsphere, wherein the target polynucleotide comprises a label capable of being recognized and/or otherwise bound by a receptor. The hybridized target is contacted with a receptor, which may comprise multiple sites capable of binding the label on the target polynucleotide, and the receptor is contacted with a ligand that may comprise binding capability to a plurality of the receptors. The presence of the complexed ligand to its receptor and requisite hybridized target then may be detected, for example, by detecting the presence of a detectable label on at least one of the receptor and the ligand. In preferred embodiments, after complexing the ligand to the receptor complexed to the hybridized target, the ligand is contacted with labeled receptor molecules, and the labeled receptor molecules complexed to the ligand are detected. This permits the detectable signal to be enhanced and more easily detected.

Compositions of the present invention are also provided wherein the compositions comprise a target polynucleotide comprising at least one label; a receptor; and a ligand, which may comprise at least one label. In one embodiment, the ligand is an antibody to the receptor, and the receptor is streptavidin or avidin.

In another embodiment, there is provided a microsphere comprising immobilized thereon at least one oligonucleotide probe hybridized to a labeled target polynucleotide, wherein the label on the target is complexed with at least one receptor, which in some embodiments comprises multiple sites capable of binding the label, and wherein the receptor is complexed to at least one ligand, said ligand being labeled and generating an amplified signal.

In one embodiment, the hybridizing of a target polynucleotide to an oligonucleotide probe is conducted in a hybridization solution comprising a buffer.

In particular embodiments, the present invention provides amplification of hybridized bead fluorescent signal using a receptor, such as streptavidin, preferably having a label, such as phycoerythrin, in conjunction with goat IgG/anti streptavidin biotinylated antibody. In specific embodiments, this amplification utilizes particular reagents and incubation conditions. Such conditions may comprise a shaking assay plate at about 500 rpm for overnight incubation at about 45° C.; hybridization/wash of assay using 0.5×TMAC buffer; and/or having about 1000 total beads in a mixture (referred to herein also as a plurality of beads) used per well. In specific embodiments, about 500 microspheres (one analyte, wherein the term “analyte” refers to a gene transcript being analyzed) up to about 100,000 microspheres (100 analytes) may be utilized in the present invention. The amplification of hybridized bead fluorescent signal using streptavidin phycoerythrin in conjunction with goat IgG/anti-streptavidin biotinylated antibody may be performed in a 0.5×TMAC buffer system or 1×MES buffer system.

Particular advantages are provided through the development of the present invention. For example, the invention replicates data from genome expression microarray measurements that facilitates assay predictive power, using a selected number of transcripts to be analyzed. That is, the present invention includes embodiments that provide an assay most consistent with genome expression microarray data. In particular embodiments, a microarray assay that measures a wide variety of genes provides information regarding genes of interest. Upon said identification, the present inventive assay provides a more focused assay to measure a particular subset of these genes of interest.

The present invention utilizes an increased sensitivity for detection of genes, even those of low abundance. For example, in particular embodiments only small amounts of input cRNA, for example, are needed, even as low as about 1.0 μg. Furthermore, using the disclosed buffer system the invention provides low % aggregation of the beads and consistently high bead counts per well. Another advantage relates to statistical methodologies utilized for oligonucleotide selection and improved methodology for analyzing assay data.

In other particular embodiments there is an algorithm for selecting optimal-performing oligonucleotide or oligonucleotides based on a previous oligonucleotide-selection assay. In particular embodiments, the oligonucleotide-selection assay is commercially available, such as the Affymetrix GeneChip assay. In particular embodiments, the number of analytes in each assay is from about 1 to about 100.

A skilled artisan recognizes that variations in parameters of the invention are well within the scope of the invention, and furthermore that a skilled artisan knows how to adjust these parameters to optimize the results. For example, the duration of assay hybridization may be a minimum of about 3 hrs but may continue for at least about 18 hrs. Also, the temperature of assay hybridization may be about 45-48° C., although depending upon the desired result other temperatures may be suitable. The amount of input polynucleotide may be as little as 1 μg to 10 μg in a complex mixture of polynucleotides, such as total RNA, mRNAs or cRNAs.

In a particular embodiment of the present invention, the amplified signal is detected using a flow cytometer, although other means to detect the amplified signal are suitable and within the scope of the present invention. The BioPlex and the Luminex 100 analyzer transfers beads from a well through a flow cytometer, where the beads are identified and read by a two laser system. The first laser identifies the analyte by exciting the fluorophores within the bead, while the second laser measures the amount of target bound to the coupled polynucleotide on the bead. This is done by excitation of the phycoerythyrin label on the target hybridized to the bead. The dynamic range of detection is expanded, allowing quantitation for low abundant and high abundant transcripts in a multiplexed platform. The recommended volume for running the assay can range from about 65-125 μl, which is the guidelines provided by the manufacturer.

The following description provides exemplary details regarding particular embodiments of the present invention, although a skilled artisan recognizes that the novel features of the invention may be modified and yet still remain within the scope of the invention.

Selection of Oligonucleotides

In a specific embodiment, the immobilized oligonucleotide probes may be selected in a non-random manner, which may also be referred to as a non-arbitrary manner. The oligonucleotide(s) may be pre-optimized, which refers to subjecting the oligonucleotide to an assay step, prior to the assay step(s) of the present invention, to determine its suitability for the inventive assay and/or to more narrowly focus the oligonucleotides utilized in the present inventive assay for efficiency and/or economic purposes. For example, one or more oligonucleotides may be subjected to a hybridization-based assay wherein a sample comprising a plurality of polynucleotides are provided to the one or more oligonucleotides, and upon detection of hybridization it is determined for a given parameter (such as a particular one or more gene sequences) which oligonucleotide(s) provided the best signal. In specific embodiments, the hybridization signal for the parameter is referred to as a hybridization fingerprint. From this hybridization fingerprint, it is determined which oligonucleotide(s) is best suited for the inventive assay described herein. In particular embodiments, this determination comprises using an algorithm.

In a specific embodiment, the algorithm comprises three main components. These are for selection of probes for a gene that varies across experimental conditions, a gene that remains constant across experimental conditions (such as a “housekeeping gene”), or genes used in assessing quality of the experiment, such as GAPDH (3′ end) or GAPDH (middle).

In a specific embodiment, the invention utilizes for the algorithm results from a prior microarray study, including the gene expression values (signal values) as well as the individual oligonucleotide probe level intensities from all microarrays in the experiment. A typical study will have one or more variable conditions, such as dose levels, chemically active agents, durations of exposure, and so forth. One or more such studies provide the data on which the probe selection is based.

For genes that vary across variable conditions, the selection is based on a measure of correlation between the gene expression value and the probe level intensities. For each probe, the measure is computed both with and without subtraction of the mismatch intensities. Also evaluated is each pair of probes, each triplet of probes, and each quadruplet of probes, since the inclusion of more than one probe (with or without its corresponding mismatch probe) may result in a better correlation. In the evaluation of doublet, triplet, and quadruplet probes, the probe sequences are examined to determine the amount overlap. For example, the best triplet may be marginally better than the best doublet, and the triplet consists of that doublet with the addition of one overlapping probe. In this case, the addition of the overlapping probe may not provide significant additional benefit.

For the embodiments utilizing genes that do not vary across experimental conditions, an objective is to minimize a measure of variability that captures the signal to noise ratio. Specifically, a Relative Standard Deviation (RSD) is used, which is expressed as the ratio of the standard deviation to the mean. This is evaluated for each Perfect Match (PM) probe, using the probe level intensities for each probe, and each “Perfect Match-Mismatch” (PM-MM) pair, using the difference of the Perfect Match and Mismatch (MM) probe level intensities. The probe having the lowest RSD is chosen.

For the embodiments that utilize genes for assessing the quality of the experiment, one measure of quality is the 3′/5′ ratio, calculated from the probe sets for GAPDH (3′ end) and GAPDH (5′ end). This ratio can vary from one microarray to the next. The ratios are calculated for each pair of probes p1 and p2, where p1 is chosen from the probe set for GAPDH (3′ end) and p2 is chosen from the probe set for GAPDH (5′ end).

Thus, in a specific embodiment, an algorithm is utilized in the present invention that has at least one of the following selection criteria: (a) selection of a PM probe with the highest measure of correlation with the signal value (this involves examination of the correlation plots to ensure that the correlation measure is not influenced by outliers; (b) select the PM and MM probe pair whose scaled (or unscaled) PM-MM probe level values have the highest measure of correlation with the signal value; (c) select the pair of probes (from two different probe sets) whose ratio best correlates with the signal ratio; and/or (d) select the PM probe having the smallest measure of variability (specifically, Relative Standard Deviation).

The algorithm may utilize at least one of the following selection criteria: (a) selecting at least one perfect match pre-optimized oligonucleotide, wherein the selected at least one perfect match pre-optimized oligonucleotide has an acceptable measure of correlation with a standard gene expression value; (b) selecting at least one perfect match and minus mismatch pre-optimized oligonucleotide pair, wherein within a pair the selected at least one perfect match pre-optimized oligonucleotide minus the mismatch pre-optimized oligonucleotide has an acceptable measure of correlation with a standard gene expression value; (c) selecting at least one pair of pre-optimized oligonucleotides from different pre-optimized oligonucleotide sets, wherein the ratio of signals in the pre-optimized oligonucleotides in the at least one pair of pre-optimized oligonucleotides has an acceptable correlation with a standard signal ratio; and (d) selecting at least one perfect match pre-optimized oligonucleotide, wherein the perfect match pre-optimized oligonucleotide has an acceptable relative standard deviation.

Regarding the term “acceptable level of correlation,” one of skill in the art recognizes that it is preferred to use the highest level of correlation, but that other substantially similar correlation values would also work in the invention. A skilled artisan recognizes that there are different ways to measure correlation, including Pearson's r, Spearman rank correlation, and various parametric, nonparametric, and robust alternatives. A skilled artisan is aware that the term “parametric” refers to being based on estimating a specific correlation parameter in a model; the term “nonparametric” refers to being based on ranks or permutation methods; and the term “robust” refers to methods that are less sensitive to outlier data.

The term “standard gene expression value,” as used herein, refers to a value obtained from at least one prior microarray output. The term applies to platforms and assays of all kinds, although in specific embodiments it is a standard signal value (also referred to as an average difference value) of an Affymetrix® GeneChip® microarray assay.

The term “standard signal ratio” as used herein, refers to the weighted sum of a ratio of signal from each of a pair of oligonucleotides.

Ligands

The ligand may be any chemical substance that comprises capability of recognizing and/or binding to a receptor. Preferably, the amplification activity comprises a plurality of ligands capable of binding to a receptor. The labels in and/or at the end of the target polynucleotide, such as the exemplary RNA polynucleotides, may be capable of binding the receptor, for example, via non-covalent specific binding interactions.

In one embodiment, the ligand may comprise an antibody. As used herein, the term “antibody” refers to an immunoglobulin molecule or a fragment thereof having the ability to specifically bind to a particular antigen. The antibody may be an anti-receptor antibody specific for the receptor used in the assay. Thus, the antibody may be capable of specifically binding the receptor as the antigen. Antibodies and methods for their manufacture are well known in the art of immunology. The antibody may be produced, for example, by hybridoma cell lines, by immunization to elicit a polyclonal antibody response, and/or by recombinant host cells that have been transformed with a recombinant DNA expression vector that encodes the antibody. Antibodies include but are not limited to immunoglobulin molecules of any isotype (IgA, IgG, IgE, IgD, IgM), and/or active fragments including Fab, Fab′, F(ab′)₂, Facb, Fv, ScFv, Fd, V_{H} and V_{L}. Antibodies include but are not limited to single chain antibodies, chimeric antibodies, mutants, fusion proteins, humanized antibodies and/or any other modified configuration of an immunoglobulin molecule that comprises an antigen recognition site of the required specificity.

The ligand preferably comprises at least one label and, in some embodiments, a plurality of labels. Preferably, the labels are covalently attached to the ligand. For example, in one embodiment, the label comprises biotin, the receptor is avidin or streptavidin, and the ligand is an anti-streptavidin antibody. In a specific embodiment, for example, a plurality of biotin molecules, e.g., about 3-10 biotin molecules, are covalently attached to the antibody.

The preparation of antibodies including antibody fragments and other modified forms is described, for example, in “Immunochemistry in Practice,” Johnstone and Thorpe, Eds., Blackwell Science, Cambridge, Mass., 1996; “Antibody Engineering,” 2^{nd} edition, C. Borrebaeck, Ed., Oxford University Press, New York, 1995; “Immunoassay”, E. P. Diamandis and T. K. Christopoulos, Eds., Academic Press, Inc., San Diego, 1996; “Handbook of Experimental Immunology,” Herzenberg et al., Eds, Blackwell Science, Cambridge, Mass., 1996; and “Current Protocols in Molecular Biology” F. M. Ausubel et al., Eds., Greene Pub. Associates and Wiley Interscience, 1987, the disclosures of which are incorporated herein. A wide variety of antibodies also are available commercially.

Amplification Using Antibodies

In one embodiment, a method is provided for detecting hybridization of a target polynucleotide, such as an RNA polynucleotide, to an oligonucleotide probe, such as an oligonucleotide linked to a microsphere. The oligonucleotide is preferably immobilized on the surface of the microsphere. In one embodiment, a label is complexed, preferably by covalent attachment, to the target polynucleotide.

In an assay, the immobilized oligonucleotide is contacted, for example, sequentially, with the target polynucleotide comprising at least one label; a receptor comprising one or more sites capable of binding the label; and an anti-receptor antibody comprising one or more labels that are preferably covalently attached to the antibody. If hybridization of the oligonucleotide probe to the target polynucleotide has occurred, then a complex is formed of at least one label of the target polynucleotide, the receptor and the antibody. The resulting complex is detected, e.g., by providing and detecting a detectable label on the antibody, or by contacting the complexed antibody with, and detecting, labeled detectable molecules of a receptor that are capable of binding to at least one label molecule on the antibody. Detection of the label thus provides a positive indicator of the hybridization of the nucleic acid target and the probe and is amplified thereby these methods.

In one embodiment, the label and receptor are biotin and streptavidin, respectively. In this embodiment, there is provided a method of determining the hybridization of a target polynucleotide with an immobilized oligonucleotide probe. A labeled target polynucleotide is provided. In some embodiments, the method comprises: contacting the immobilized oligonucleotide probe, for example in succession, with the following: an exemplary biotinylated target polynucleotide; exemplary streptavidin; an exemplary biotinylated anti-streptavidin antibody comprising a plurality of biotins; and labeled streptavidin molecules. The streptavidin is labeled with a detectable label, such as a fluorescent label. In this embodiment, the binding by hybridization of the target polynucleotide to the probe may be detected with high sensitivity. Upon hybridization of the oligonucleotide probe and the target, the target includes only one or a few biotin moieties to which streptavidin may be complexed. In some embodiments, upon complexation of streptavidin with the biotinylated target polynucleotide, the number of biotin molecules is greatly amplified. In this same embodiment, upon complexing labeled streptavidin to the biotins on the antibody, the number of detectable labels is greatly amplified, thus greatly enhancing the sensitivity of the assay.

Labels and Detection Thereof

In a specific embodiment, a label is provided on or with a component of the invention described herein. A skilled artisan recognizes that this label may be detectable, or, alternatively, the label serves the purpose of a binding entity for another component, such as a receptor, and may not be detected, such as directly detected. In one embodiment, the label for the target polynucleotide, such as the RNA polynucleotide, is biotin, and the receptor is avidin or streptavidin. For example, in the embodiment wherein the ligand is an antibody, biotin may be covalently attached to the antibody. For example, the antibody may be an anti-streptavidin antibody comprising a plurality of biotin molecules covalently attached to the antibody. In an assay, after complexing of the antibody to a streptavidin receptor bound to the biotinylated target polynucleotide, the antibody may be contacted with labeled streptavidin, thereby to complex a plurality of labeled streptavidin molecules to the antibody, and the labeled streptavidin molecules complexed to the antibody then may be detected, thus providing signal amplification in the assay.

The label may be provided on the ligand, the receptor and/or the target polynucleotide. Examples of labels include fluorescent labels, chemiluminescent labels, and inorganic labels, such as gold, as well as enzymatic labels.

Labels may be referred to as being detectable, for example, by chromogenic detection, chemiluminescent detection and fluorescent detection. Exemplary labels include marker enzymes such as alkaline phosphatase, β-galactosidase or horseradish peroxidase, which are detected using a chromogenic substrate. For example, alkaline phosphatase may be detected using 5-bromo-4-chloro-3-indolyl phosphate or nitroblue tetrazolium salt.

In one preferred embodiment, the avidin or streptavidin may be complexed with a fluorescent label, such as phycoerythrin, in particular embodiments. In one embodiment, the detectable streptavidin that may be used is streptavidin phycoerythrin, which is commercially available, for example, from Molecular Probes (Eugene, Oreg.). Biotinylated anti-streptavidin antibody is available, for example, from Vector Laboratories (Burlingame, Calif.).

Avidin-biotin systems have been developed for use in a variety of detection assays. Methods for the detection and labeling of nucleic acids in biotin systems are described, for example, in “Nonradioactive Labeling and Detection Systems”, C. Kessler, Ed., Springer-Verlag, New York, 1992, pp. 70-99; and in “Methods in Nonradioactive Detection,”, G. Howard, Ed., Appleton and Lange, Norwalk, Conn. 1993, pp. 11-27 and 137-150.

Fluorescent tags such as phycoerythrin, fluorescein, rhodamine, and resorufin, and derivatives thereof, as well as coumarins such as hydroxycoumarin, may be used in the invention. Additionally, fluorescence resonance energy transfer may be measured, as described in Cardullo, Nonradiative Fluorescence Resonance Energy Transfer in “Nonradioactive Labeling and Detection of Biomolecules”, C. Kessler, Ed., Springer-Verlag, New York, 1992, pp. 414-423, the disclosure of which is incorporated herein. Alternatively, inorganic labels may be used in the invention, such as colloidal gold particles or ferritin. The use of colloidal gold particles as labels is described, for example, in Van de Plas and Leunissen, Colloidal Gold as a Marker in Molecular Biology: The Use of Ultra-Small Gold Particles, in “Nonradioactive Labeling and Detection of Biomolecules”, C. Kessler, Ed., Springer-Verlag, New York, 1992, pp. 116-126, the disclosure of which is incorporated herein.

Reagents for labeling streptavidin or avidin with a fluorescent tag are commercially available. For example, the exemplary reagents, 5(6)-Carboxyfluorescein-N-hydroxysuccinimide ester (FLUOS), 7-amino-4-methyl-coumarin-3-acetic acid-N′-hydroxysuccinimide ester (AMCA, acitvated) and fluorescein isothiocyanate (FITC) are commercially available from Boehringer Mannheim, Indianapolis, Ind. Methods for fluorescently labeling proteins with fluorescent labels, and methods for detection of the fluorescent labels, are described in Howard, G., Labeling Proteins with Fluorochromes, in “Methods in Nonradioactive Detection,”, G. Howard, Ed., Appleton and Lange, Norwalk, Conn. 1993, pp. 39-68, the disclosure of which is incorporated herein. Additionally, there are a variety of commercially available labeled streptavidin and avidin molecules. Non-limiting examples include streptavidin-gold, streptavidin-fluorochrome, streptavidin-AMCA, streptavidin-fluorescein, streptavidin-phycoerythrin (SAPE), streptavidin-sulforhodamine 101, avidin-FITC and avidin-Texas red®, which are commercially available from Boehringer Mannheim, Indianapolis, Ind.

Methods available in the art for attaching labels, to polynucleotides are known. In one embodiment, nucleic acids having a label covalently attached can be synthesized using a DNA synthesizer and standard phosphoramidite reagents. For example, biotin phosphoramidites for direct labeling of synthetic oligonucleotides may be used. Biotin phosphoramidites are commercially available from Glen Research Corporation, Sterling, Va.

In one embodiment, in the case where the label is biotin, biotinylated DNA targets can be prepared using nick translation and random primer extension, while biotinylated RNA targets can be synthesized by in vitro transcription using an RNA polymerase. Biotinylated deoxyribonucleoside triphosphates and ribonucleoside triphosphates have been used for the enzymatic preparation of biotinylated DNA and biotinylated RNA. Exemplary methods are disclosed in detail in Rashtchian and Mackey, Labeling and Detection of Nucleic Acids, in “Nonradioactive Labeling and Detection of Biomolecules”, C. Kessler, Ed., Springer-Verlag, New York, 1992, pp. 70-84. The concentration of biotin molecules may be increased by the use of a psoralen biotin reagent, as described in Levenson et al, Methods Enzymol., 184:577-583 (1990); and Cimono et al., Ann. Rev. Biochem. 54:1151-1193 (1985), the disclosures of each of which are incorporated herein. Background hybridization may be reduced by HPLC purification of biotinylated target nucleic acids.

Labels, such as biotins, may be attached to ligands, such as polymers, including antibodies, using methods available in the art. Exemplary methods are disclosed in detail in Bayer and Wilchek, Labeling and Detection of proteins and Glycoproteins, in “Nonradioactive Labeling and Detection of Biomolecules”, C. Kessler, Ed., Springer-Verlag, New York, 1992, pp. 91-100 and referenced cited therein, the disclosures of which are incorporated herein by reference. Furthermore, biotinylated antibodies, such as biotinylated anti-streptavidin molecules, are available commercially, for example, from Vector Laboratories (Burlingame, Calif.).

Label Receptor Pairs

As used herein, the phrase “label-receptor pair” refers to a label and receptor that are chemical moieties capable of recognizing and binding to each other. The label and receptor can be any moieties that are capable of recognizing and binding to each other to form a complex. In some embodiments, the label and receptor may interact via the binding of a third intermediary substance. Typically, the label and receptor constituting the label-receptor pair are binding molecules that undergo a specific noncovalent binding interaction with each other. The label and receptor can be naturally occurring or artificially produced, and optionally may be aggregated with other species.

Preferably, a label-receptor pair includes a receptor that is capable of binding a plurality, e.g., 2, 3, 4 or more, molecules of the label. In one preferred embodiment, the label-receptor pair is biotin-avidin, respectively, or biotin-streptavidin, respectively. The vitamin biotin is detected by binding of the indicator protein avidin, isolated from egg white, or streptavidin, isolated from Streptomyces avidinii bacteria. Avidin and streptavidin have four high affinity binding sites for biotin with a binding constant of about K=10¹⁵ mol⁻¹. Kessler, Overview of Nonradioactive Labeling Systems in “Nonradioactive Labeling and Detection of Biomolecules”, C. Kessler, Ed., Springer-Verlag, New York, 1992, pp. 27-34, the disclosure of which is incorporated herein.

Ligands used in the assay methods disclosed herein can be attached to any of a variety of members of label-receptor binding pairs available in the art. In one preferred embodiment, in nucleic acid hydridization assays using an immobilized oligonucleotide capable of hybridizing to a target polynucleotide, the target polynucleotide comprises a label constituting a member of a label-receptor binding pair. Additionally, the ligand may include a plurality of labels. Preferably, the receptor of the label-receptor pair is capable of binding to more than one molecule of label. For example, the label may be biotin and the receptor may be avidin or streptavidin, each of which are capable of binding four molecules of biotin. Hybridization of the target polynucleotide to the probe oligonucleotide may be detected by detecting binding of the label of the target polynucleotide to the receptor, and further by binding of the receptor to the ligand and further by binding of a labeled receptor that binds to the ligand. The ligand is detected, e.g., by providing a label on the ligand, or by complexing the ligand with a plurality of molecules of labeled receptor.

Hybridization

A skilled artisan recognizes that the ability of two nucleic acids, each having at least one single stranded region, to hybridize to each other depends upon a variety of aspects, including the degree of complementarity between the single stranded region(s) of the two molecules and the stringency of the hybridization reaction conditions. In a specific embodiment, the hybridization is between an immobilized oligonucleotide probe and an input target polynucleotide, such as a RNA polynucleotide, for example an mRNA. In specific embodiments, hybridization conditions are such that there is complete complementarity between the entire sequence of the immobilized oligonucleotide and at least a portion of a target polynucleotide.

Methods for conducting nucleic acid hybridization assays have been well developed in the art. Hybridization assay procedures and conditions will vary depending on the application and are selected in accordance with the general binding methods known in the art.

The present invention, in some embodiments, utilizes particular buffers and buffer concentrations. In a specific embodiment, 0.5×TMAC, made from 1×TMAC, is utilized for suspending sample polynucleotide and/or hybridization buffer. A skilled artisan recognizes that 1×TMAC comprises 3M TMAC, 0.1% Sarcosyl, 50 mM Tris-HCl pH8.0, and 4 mM EDTA pH8.0.

For some applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the oligonucleotide probe and target polynucleotide. It is generally appreciated that conditions can be rendered more stringent by the addition of increasing amounts of formamide.

For certain applications, it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results, and a skilled artisan is aware how to perform such manipulations.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

Other nucleic acid hybridization buffers commonly used in the art include phosphate and TRIS buffers, for example, at a pH of about 6 to 8. In one embodiment, a standard saline phosphate ethylenediaminetetraacetic acid (“SSPE”) buffer is used. An exemplary phosphate buffer includes: 0.06M H₂PO₄/HPO₄, 1M Na⁺, 0.006M EDTA (ethylenediaminetetraacetic acid), 0.005% Triton®, at a pH of about 6.8, referred to herein as “6×SSPE-T”.

In some embodiments of the present invention, a method is provided for conducting nucleic acid hybridization assays, wherein the hybridization solution comprises a sulfonate buffer. Sulfonate hybridization buffers include 2[N-morpholino]ethanesulfonic acid (“MES”) and 3[N-morpholino]propanesulfonic acid) (“MOPS”). In one embodiment, the hybridization assay using a sulfonate buffer may be conducted with nucleic acid probes immobilized on a solid surface, such as a microsphere. The solid surface may be, for example, coated with a silane coating prior to immobilization of the nucleic acid probes. The hybridization assay in a solution comprising a sulfonate buffer may be conducted, for example, at a temperature of about 25 to 70° C., for example, at least about 35° C., or 45° C. or more, and over a time period of, for example, about 10 minutes to about 5 hours or more, e.g., about 16 hours or more. The sulfonate buffer may be used, for example in gene expression hybridization assays and other hybridization assays.

For example, the hybridization buffer may include about 0.01 M to about 2 M MES or more, e.g., about 0.25 M MES, at a pH, for example, of about 6 to 7. In one embodiment, the MES buffer includes: 0.25M MES, 1M Na⁺, and 0.005% Triton® X-100, at a pH of about 5.5-6.7, e.g. 6.7. The hybridization may be conducted, for example, at about 25 to 70° C., for example, about 45° C. Optionally, the buffer may be filtered prior to use, for example, through a 2 μm filter. The nucleic acid hybridization buffers may further include surfactants, such as Tween-20 and Triton-X100, as well as additives such as anti-foaming agents.

Kits

In an embodiment of the present invention, kits are provided for amplifying a signal from a bead-based oligonucleotide hybridization assay that may include in suitable packaging at least one of the following materials: microspheres, immobilized oligonucleotide probes separately and/or on the microspheres, receptors, labels, and ligands, which may be provided comprising labels. Reagents to detect a label or detect amplification of a label may also be included in the kit. The reagents may be, for example, in separate containers in the kit. The kit may also include hybridization buffers, wash solutions, negative and positive controls and written instructions for performing the assay.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Exemplary Assay Protocol

The present example provides an exemplary assay protocol, wherein microspheres comprising oligonucleotides are subjected to a sample comprising cRNA polynucleotides, hybridization incubation between an oligonucleotide and a target polynucleotide occurs, and the complex is stained with a receptor followed by staining of the receptor with a ligand and then staining of the ligand with a label.

A skilled artisan recognizes that buffers are utilized during particular steps of methods described herein. For example, a buffer may be used to suspend the plurality of target polynucleotides, such as RNA polynucleotides. Although a skilled artisan is aware that conditions for incubations, hybridizations, and the like may be altered in accordance with the requirements of the procedure, the following text describes exemplary useful assay conditions.

1. Dilution of Beads Sets (A Bead quality control protocol may be used for determining concentration of beads after coupling. For example, a bead(s) is coupled to at least one oligonucleotide and subjected to the present assay in serial dilution to determine the preferable amount of oligonucleotide coupled to bead. A second assay is performed in multiplex to determine cross-hybridization probability to beads representing other analytes.)
a) Use 0.5×TMAC buffer volume dependent on amount of samples being processed and number of beads;
b) Standard concentration of beads is 10⁷ beads per ml;
c) 40 μl of diluted bead mixture is added to each well (˜1000 beads per well);
   To generate the bead mixture in 1C having 800 μl in volume: 2 μl each bead used in 5Plex and 790 μl 0.5×TMAC Hybridization (Hyb) Buffer; or 2 μl each bead used in 20Plex and 760 μl 0.5×TMAC Hybridization Buffer.

2. Target cRNA Calculation (Note cRNA is fragmented at a concentration of 0.5 μg/μl)
a) Dilutions are performed with 0.5×TMAC Hyb buffer comprising M13oligo;
b) Determination of how many samples are run, including blanks;
c) 20 μl of cRNA is added to each well (2 μg);
   Dilution of M13 oligo at a 2×10⁶ dilution (M13 stock solution=1 mM): 2 μl of 1 nM into 998 μl TE=2 μM; 2 μl of 2 uM into 198 μl TE=20 nM; and 2.5 μl of 20 nM into 397.5 μl TE=125 pM working solution.

For Duplicate wells, the target cRNA is calculated as follows: For 5 μg cRNA per well, use 25 μl stock cRNA (0.5 ug/ul) and 25 μl 0.5×TMAC Hyb buffer containing 100 attomole (amol) of M13 (15 μl M13 working solution to 235 μl 0.5×TMAC Hyb Buffer). For 2.5 μg cRNA per well, use 12.5 μl Stock cRNA (0.5 μg/μl) and 37.51 μl 0.5×TMAC Hyb buffer containing 100 amol of M13 (10 μl M13 working solution to 240 μl 0.5×TMAC Hyb Buffer). For 2.0 μg cRNA per well, use 10 μl Stock cRNA (0.5 μg/μl) and 40 μl 0.5×TMAC Hyb buffer containing 100 amol of M13 (8 μl M13 working solution to 242 μl 0.5×TMAC Hyb Buffer).

3. Reagents
a) 1×TMAC=3M TMAC, 0.1% Sarcosyl, 50 mM Tris-HCl pH8.0, 4 mM EDTA pH 8.0, wherein 0.5×TMAC=made from 1×TMAC;
b) PBS-BSA wash Buffer=9.7 ml PBS+330 μl 30% BSA; and

c) Volumes of stains are determined based upon 200 μl per sample for StreptAv, 100 μl per sample for Antibody.

| StreptAv-PE | | GoatIgG | | Anti-StAv | |
|---|---|---|---|---|---|
| Stock Con. | 1 mg/ml | Stock Con | 10 mg/ml | Stock Con | 0.5 mg/ml |
| Final Con | 20 μg/ml | Final Con | 100 μg/ml | Final Con | 5 μg/ml |

4. Hybridization
1. Add 20 μl of diluted probes according to step 2 above;
2. Add 40 μl of bead mix to each well according to step 1 above;
3. Incubate at 95° C. for 2 minutes;
4. Transfer plate to thermo mixer, cover and hybridize overnight at 45° C. shaking at 500 rpm;
5. Spin samples in centrifuge at 2250 g for 2 min, flick and tap off solution;
6. Wash beads with 100 μl of 0.5×TMAC; shake 500 rpm at 25° C. for 2 min;
7. Spin samples in centrifuge at 2250 g for 2 min, flick and tap off solution;
8. Wash beads with 100 μl of PBS-BSA; shake 500 rpm at 25° C. for 2 min;
9. Spin samples in centrifuge at 2250 g for 2 min, flick and tap off solution;
10. Add 100 μl of StreptAvidin-PE (StAv-PE) stain mix; shake 500 rpm at 25° C. for 10 min;
11. Spin samples in centrifuge at 2250 g for 2 min, flick and tap off solution;
12. Wash beads with 100 μl of PBS-BSA; shake 500 rpm at 25° C. for 2 min;
13. Spin samples in centrifuge at 2250 g for 2 min, flick and tap off solution;
14. Add 100 μl of Second Stain (anti-StAv and nGtIgG); shake 500 rpm at 25° C. for 10 min;
15. Spin samples in centrifuge at 2250 g for 2 min, flick and tap off solution;
16. Wash beads with 100 μl of PBS-BSA; shake 500 rpm at 25° C. for 2 min;
17. Spin samples in centrifuge at 2250 g for 2 min, flick and tap off solution;
18. Add 100 μl of Third Stain (StAv-PE); shake 500 rpm at 25° C. for 10 min;
19. Spin samples in centrifuge at 2250 g for 2 min, flick and tap off solution;
20. Wash beads with 100 μl of PBS-BSA; shake 500 rpm at 25° C. for 10 min;
21. Spin samples in centrifuge at 2250 g for 2 min, flick and tap off solution;
22. Resuspend in 65 μl PBS-BSA and read on Bioplex; and
23. Shake plate very well before running on bioplex.

### Example 2

### Amplification of Signal in Oligonucleotide Assay

The amplification of a signal from a hybridization-based oligonucleotide assay is performed as described herein. Table 1 illustrates a titration assay for particular cRNA sequences (and the control M13) at different hybridization times and for different sample parameters (wherein Low, Med, and High refers to respective estradiol levels from a biological sample). The fold change is calculated based on a ratio of sample output over vehicle output. Compared to known methods in the art, the present invention provides at least about 100-fold amplification of signal.

TABLE 1
Titration Assay for Specific Oligonucleotides

| | fmol | 018 M13 | 019 ICAP | 020 CYP17 | 021 11BHSD7 |
|---|---|---|---|---|---|
| 1 hr Hyb | 10 | 25815 | 26079 | 26017 | 26108 |
| | 1 | 19651 | 20527 | 16499 | 22626 |
| | 0.1 | 2536 | 2459 | 1817 | 3333 |
| | 0.01 | 179 | 188 | 152 | 256 |
| | 0.001 | 22 | 26 | 23 | 30 |
| | 0.0001 | 9 | 11 | 14 | 13 |
| | 0 | 7 | 9 | 12 | 9 |
| | 0 | 6 | 10 | 10 | 9 |
| | Vehicle | 38 | 273 | 848 | 65 |
| | Low | 31 | 498 | 332 | 59 |
| | Med | 33 | 3083 | 122 | 81 |
| | High | 24 | 6268 | 93 | 103 |
| 3 hr Hyb | 10 | 24707 | 25051 | 25024 | 25094 |
| | 1 | 21779 | 22560 | 18487 | 23072 |
| | 0.1 | 3122 | 2765 | 1964 | 4115 |
| | 0.01 | 265 | 221 | 172 | 300 |
| | 0.001 | 30 | 27 | 26 | 33 |
| | 0.0001 | 11 | 13 | 13 | 15 |
| | 0 | 9 | 11 | 14 | 12 |
| | 0 | 7 | 10 | 10 | 9 |
| | Vehicle | 37 | 365 | 1445 | 88 |
| | Low | 38 | 952 | 628 | 111 |
| | None | 0 | 0 | 0 | 0 |
| | High | 25 | 12145 | 123 | 197 |
| 19 hr Hyb | 10 | 27340 | 27412 | 27405 | 27394 |
| | 1 | 24048 | 24466 | 21826 | 25314 |
| | 0.1 | 3901 | 3504 | 2615 | 5061 |
| | 0.01 | 344 | 311 | 261 | 450 |
| | 0.001 | 63 | 70 | 60 | 77 |
| | 0.0001 | 21 | 26 | 27 | 29 |
| | 0 | 20 | 26 | 30 | 24 |
| | 0 | 15 | 20 | 22 | 22 |
| | Vehicle | 569 | 834 | 3701 | 234 |
| | Low | 983 | 2282 | 2661 | 285 |
| | Med | 1992 | 10286 | 2424 | 511 |
| | High | 1669 | 22404 | 2328 | 792 |
| Fold change | 1 hr Low | 0.82 | 1.82 | 0.39 | 0.90 |
| | 1 hr Med | 0.86 | 11.29 | 0.14 | 1.25 |
| | 1 hr Hi | 0.63 | 22.96 | 0.11 | 1.58 |
| | 3 hr Low | 1.0 | 2.6 | 0.4 | 1.3 |
| | 3 hr Hi | 0.7 | 33.3 | 0.1 | 2.2 |
| | 19 hr Low | 1.7 | 2.7 | 0.7 | 1.2 |
| | 19 hr Med | 3.5 | 12.3 | 0.7 | 2.2 |
| | 19 hr Hi | 2.9 | 26.9 | 0.6 | 3.4 |
| | fmol | 033 PPIB9 | 034 STAR | 035 UOSPT | 036 PECOA |
| 1 hr Hyb | 10 | 25679 | 26038 | 26103 | 25802 |
| | 1 | 20308 | 21805 | 19047 | 19818 |
| | 0.1 | 3018 | 2975 | 2479 | 2540 |
| | 0.01 | 255 | 238 | 204 | 205 |
| | 0.001 | 35 | 29 | 37 | 27 |
| | 0.0001 | 16 | 12 | 28 | 10 |
| | 0 | 12 | 9 | 21 | 8 |
| | 0 | 13 | 10 | 19 | 7 |
| | Vehicle | 993 | 164 | 218 | 31 |
| | Low | 839 | 130 | 192 | 29 |
| | Med | 819 | 55 | 6586 | 87 |
| | High | 654 | 51 | 3541 | 47 |
| 3 hr Hyb | 10 | 24228 | 24795 | 25012 | 24913 |
| | 1 | 23166 | 23551 | 23002 | 23307 |
| | 0.1 | 4563 | 5170 | 4392 | 4999 |
| | 0.01 | 437 | 399 | 410 | 411 |
| | 0.001 | 52 | 44 | 55 | 45 |
| | 0.0001 | 21 | 16 | 46 | 14 |
| | 0 | 14 | 10 | 27 | 10 |
| | 0 | 14 | 8 | 27 | 10 |
| | Vehicle | 2439 | 276 | 388 | 38 |
| | Low | 2718 | 274 | 439 | 50 |
| | None | 0 | 0 | 0 | 0 |
| | High | 1689 | 55 | 8790 | 108 |
| 19 hr Hyb | 10 | 27100 | 27427 | 27519 | 27254 |
| | 1 | 25307 | 26149 | 26082 | 26605 |
| | 0.1 | 5716 | 7012 | 7582 | 8774 |
| | 0.01 | 641 | 631 | 683 | 822 |
| | 0.001 | 102 | 95 | 128 | 109 |
| | 0.0001 | 34 | 28 | 94 | 38 |
| | 0 | 28 | 23 | 55 | 26 |
| | 0 | 25 | 21 | 52 | 22 |
| | Vehicle | 6510 | 881 | 1093 | 127 |
| | Low | 6803 | 783 | 1298 | 160 |
| | Med | 3599 | 153 | 23613 | 626 |
| | High | 4573 | 136 | 21926 | 434 |
| Fold change | 1 hr Low | 0.84 | 0.79 | 0.88 | 0.94 |
| | 1 hr Med | 0.82 | 0.33 | 30.21 | 2.81 |
| | 1 hr Hi | 0.66 | 0.31 | 16.24 | 1.52 |
| | 3 hr Low | 1.1 | 1.0 | 1.1 | 1.3 |
| | 3 hr Hi | 0.7 | 0.2 | 22.7 | 2.8 |
| | 19 hr Low | 1.0 | 0.9 | 1.2 | 1.3 |
| | 19 hr Med | 0.6 | 0.2 | 21.6 | 4.9 |
| | 19 hr Hi | 0.7 | 0.2 | 20.1 | 3.4 |

| | fmol | 037 FN3M1 | 038 CDK4 | 039 FSKREG | 051 SPP1 |
|---|---|---|---|---|---|
| 1 hr Hyb | 10 | 25862 | 25869 | 25880 | 25857 |
| | 1 | 18902 | 21614 | 19631 | 20076 |
| | 0.1 | 2365 | 3436 | 2398 | 2508 |
| | 0.01 | 193 | 291 | 212 | 204 |
| | 0.001 | 26 | 49 | 33 | 31 |
| | 0.0001 | 14 | 46 | 18 | 17 |
| | 0 | 10 | 23 | 15 | 14 |
| | 0 | 10 | 21 | 16 | 14 |
| | Vehicle | 585 | 523 | 87 | 183 |
| | Low | 619 | 405 | 74 | 92 |
| | Med | 923 | 299 | 112 | 74 |
| | High | 1043 | 260 | 101 | 52 |
| 3 hr Hyb | 10 | 24893 | 25040 | 24978 | 24857 |
| | 1 | 21455 | 23418 | 23171 | 22743 |
| | 0.1 | 2694 | 6404 | 4664 | 4149 |
| | 0.01 | 225 | 547 | 380 | 362 |
| | 0.001 | 31 | 75 | 53 | 44 |
| | 0.0001 | 15 | 75 | 27 | 20 |
| | 0 | 12 | 32 | 24 | 16 |
| | 0 | 11 | 28 | 23 | 17 |
| | Vehicle | 1019 | 1025 | 118 | 335 |
| | Low | 1436 | 950 | 132 | 185 |
| | None | 0 | 0 | 0 | 0 |
| | High | 2438 | 546 | 165 | 90 |
| 19 hr Hyb | 10 | 27492 | 27385 | 27326 | 27356 |
| | 1 | 24048 | 26607 | 25914 | 25538 |
| | 0.1 | 3533 | 9638 | 6859 | 5548 |
| | 0.01 | 315 | 1032 | 653 | 537 |
| | 0.001 | 66 | 155 | 120 | 94 |
| | 0.0001 | 28 | 159 | 46 | 35 |
| | 0 | 32 | 56 | 42 | 30 |
| | 0 | 21 | 49 | 39 | 26 |
| | Vehicle | 2861 | 3088 | 266 | 855 |
| | Low | 4383 | 2758 | 293 | 459 |
| | Med | 4640 | 1261 | 451 | 329 |
| | High | 8149 | 1560 | 420 | 259 |
| Fold change | 1 hr Low | 1.06 | 0.77 | 0.85 | 0.50 |
| | 1 hr Med | 1.58 | 0.57 | 1.28 | 0.40 |
| | 1 hr Hi | 1.78 | 0.50 | 1.16 | 0.28 |
| | 3 hr Low | 1.4 | 0.9 | 1.1 | 0.6 |
| | 3 hr Hi | 2.4 | 0.5 | 1.4 | 0.3 |
| | 19 hr Low | 1.5 | 0.9 | 1.1 | 0.5 |
| | 19 hr Med | 1.6 | 0.4 | 1.7 | 0.4 |
| | 19 hr Hi | 2.8 | 0.5 | 1.6 | 0.3 |

| | fmol | 052 C3 | 053 GFBP3 | 054 CKB | 055 HSP27 |
|---|---|---|---|---|---|
| 1 hr Hyb | 10 | 25992 | 25699 | 26258 | 25977 |
| | 1 | 17602 | 18175 | 22953 | 21368 |
| | 0.1 | 1908 | 2249 | 3634 | 3175 |
| | 0.01 | 166 | 198 | 311 | 280 |
| | 0.001 | 27 | 28 | 41 | 57 |
| | 0.0001 | 15 | 14 | 16 | 38 |
| | 0 | 15 | 13 | 16 | 32 |
| | 0 | 15 | 15 | 15 | 32 |
| | Vehicle | 111 | 183 | 416 | 339 |
| | Low | 152 | 147 | 425 | 330 |
| | Med | 7613 | 75 | 689 | 466 |
| | High | 4841 | 51 | 746 | 531 |
| 3 hr Hyb | 10 | 25008 | 24588 | 24976 | 24750 |
| | 1 | 19744 | 23142 | 23665 | 23252 |
| | 0.1 | 2276 | 4736 | 6604 | 4959 |
| | 0.01 | 187 | 430 | 570 | 455 |
| | 0.001 | 29 | 46 | 59 | 75 |
| | 0.0001 | 17 | 17 | 21 | 45 |
| | 0 | 18 | 16 | 16 | 43 |
| | 0 | 14 | 11 | 16 | 40 |
| | Vehicle | 118 | 373 | 872 | 588 |
| | Low | 215 | 379 | 1179 | 654 |
| | None | 0 | 0 | 0 | 0 |
| | High | 10178 | 85 | 2060 | 1036 |
| 19 hr Hyb | 10 | 27526 | 27446 | 27527 | 27227 |
| | 1 | 22520 | 26457 | 26693 | 25647 |
| | 0.1 | 3026 | 10424 | 9926 | 6936 |
| | 0.01 | 282 | 978 | 928 | 643 |
| | 0.001 | 64 | 131 | 131 | 141 |
| | 0.0001 | 28 | 35 | 35 | 73 |
| | 0 | 30 | 33 | 34 | 70 |
| | 0 | 23 | 25 | 27 | 64 |
| | Vehicle | 268 | 1539 | 3396 | 1369 |
| | Low | 490 | 1515 | 4279 | 1519 |
| | Med | 18208 | 449 | 4303 | 1613 |
| | High | 16433 | 356 | 7806 | 2624 |
| Fold change | 1 hr Low | 1.37 | 0.80 | 1.02 | 0.97 |
| | 1 hr Med | 68.59 | 0.41 | 1.66 | 1.37 |
| | 1 hr Hi | 43.61 | 0.28 | 1.79 | 1.57 |
| | 3 hr Low | 1.8 | 1.0 | 1.4 | 1.1 |
| | 3 hr Hi | 86.3 | 0.2 | 2.4 | 1.8 |
| | 19 hr Low | 1.8 | 1.0 | 1.3 | 1.1 |
| | 19 hr Med | 67.9 | 0.3 | 1.3 | 1.2 |
| | 19 hr Hi | 61.3 | 0.2 | 2.3 | 1.9 |

| | fmol | 056 CTSB | 057 SCYA11 | 058 PROGREC | 072 VAACTIN |
|---|---|---|---|---|---|
| 1 hr Hyb | 10 | 8072 | 25798 | 25824 | 25946 |
| | 1 | 1790 | 19984 | 19905 | 19439 |
| | 0.1 | 214 | 2681 | 3165 | 2522 |
| | 0.01 | 34 | 239 | 292 | 227 |
| | 0.001 | 14 | 34 | 40 | 46 |
| | 0.0001 | 15 | 16 | 22 | 31 |
| | 0 | 12 | 12 | 19 | 28 |
| | 0 | 13 | 13 | 20 | 27 |
| | Vehicle | 45 | 127 | 102 | 761 |
| | Low | 44 | 137 | 98 | 716 |
| | Med | 49 | 444 | 124 | 724 |
| | High | 49 | 559 | 101 | 516 |
| 3 hr Hyb | 10 | 11028 | 24558 | 24812 | 24655 |
| | 1 | 3834 | 22954 | 22985 | 23284 |
| | 0.1 | 589 | 4876 | 4919 | 4663 |
| | 0.01 | 78 | 429 | 428 | 376 |
| | 0.001 | 21 | 51 | 58 | 61 |
| | 0.0001 | 16 | 20 | 27 | 29 |
| | 0 | 18 | 15 | 25 | 27 |
| | 0 | 18 | 14 | 23 | 27 |
| | Vehicle | 63 | 170 | 149 | 1756 |
| | Low | 70 | 262 | 194 | 1911 |
| | None | 0 | 0 | 0 | 0 |
| | High | 93 | 1545 | 199 | 1251 |
| 19 hr Hyb | 10 | 12425 | 26883 | 27541 | 27062 |
| | 1 | 9882 | 25633 | 26086 | 25794 |
| | 0.1 | 3437 | 6728 | 7249 | 7001 |
| | 0.01 | 424 | 712 | 662 | 662 |
| | 0.001 | 120 | 107 | 115 | 107 |
| | 0.0001 | 42 | 30 | 43 | 44 |
| | 0 | 49 | 28 | 42 | 44 |
| | 0 | 37 | 24 | 38 | 37 |
| | Vehicle | 299 | 374 | 357 | 5891 |
| | Low | 301 | 685 | 468 | 6277 |
| | Med | 315 | 2262 | 470 | 4418 |
| | High | 498 | 5162 | 552 | 4262 |
| Fold change | 1 hr Low | 0.98 | 1.08 | 0.96 | 0.94 |
| | 1 hr Med | 1.09 | 3.51 | 1.22 | 0.95 |
| | 1 hr Hi | 1.09 | 4.42 | 0.99 | 0.68 |
| | 3 hr Low | 1.1 | 1.5 | 1.3 | 1.1 |
| | 3 hr Hi | 1.5 | 9.1 | 1.3 | 0.7 |
| | 19 hr Low | 1.0 | 1.8 | 1.3 | 1.1 |
| | 19 hr Med | 1.1 | 6.0 | 1.3 | 0.7 |
| | 19 hr Hi | 1.7 | 13.8 | 1.5 | 0.7 |

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

Thus, all documents cited are, in relevant part, incorporated herein by reference; the citation of any document is not to be construed as an admission that it is prior art with respect to the present invention.

### PATENTS

U.S. Pat. No. 6,203,989
U.S. patent Application No. 2001/0041335
U.S. patent Application No. 2002/0034753

### PUBLICATIONS

Bhalgat, M. K., Haugland, R. P., Pollack, J. S., Swan, S., Haugland, R. P. Green- and red-fluorescent nanospheres for the detection of cell surface receptors by flow cytometry, J. Immunolog. Methods 219:57-68, 1998.
Dunbar, S. A., Zee, C. A. V., Oliver, K. G., Karem, K. L., Jacobsen, J. W. Quantitative, multiplexed detection of bacterial pathogens: DNA and protein applications of the Luminex LabMAP™ system, J. Microbiolog. Meth. 53: 245-252, 2003.
Lindmo, T., Bormer, O., Ugelstad, J., and Nustad, K. Immunometric assay by flow cytometry using mixtures of two particle types of different affinity, J. Immunolog. Methods 126:183-189, 1990.
McHugh, T. M., Miner, R. C., Logan, L. H., and Stites, D. P. Simultaneous Detection of Antibodies to Cytomegalovirus and Herpes Simplex Virus by using flow cytometry and a microsphere-based fluorescence immunoassay, J. Clin. Microbiol. 26(1):1957-1961, 1988.
Spycher, M. O., Spycher-Burger, M., Spath, P. J., and Burckhardt, J. J. Human serum induced opsonization of immunoglobin G-coated polystyrene microspheres with complement components C3 and C4 as measured by flow cytometry, J. Immunolog. Methods 145:83-92, 1991.
Yang, L., Tran, D. K., Wang, X. BADGE, Beads Array for the Detection of Gene Expression, a High-Throughput Diagnostic Bioassay, Genome Research 11:1888-1898, 2001.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

Thus, while particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method for amplifying a signal for detection of a polynucleotide, comprising the steps of:
(a) providing at least one microsphere linked to at least one pre-optimized oligonucleotide;
(b) hybridizing a labeled target polynucleotide to said oligonucleotide to form an oligonucleotide/target polynucleotide complex, wherein said complex comprises a detectable signal through the binding of a receptor to the label; and
(c) providing a labeled ligand for said receptor, wherein when said ligand binds said receptor, said signal is amplified.

2. The method of claim 1, wherein the pre-optimized oligonucleotide is selected with an algorithm.

3. The method of claim 2, wherein said algorithm utilizes at least one of the following selection criteria:
(a) selecting at least one perfect match pre-optimized oligonucleotide, wherein the selected at least one perfect match pre-optimized oligonucleotide has an acceptable measure of correlation with a standard gene expression value;
(b) selecting at least one perfect match and minus mismatch pre-optimized oligonucleotide pair, wherein within a pair the selected at least one perfect match pre-optimized oligonucleotide minus the mismatch pre-optimized oligonucleotide has an acceptable measure of correlation with a standard gene expression value;
(c) selecting at least one pair of pre-optimized oligonucleotides from different pre-optimized oligonucleotide sets, wherein the ratio of signals in the pre-optimized oligonucleotides in the at least one pair of pre-optimized oligonucleotides has an acceptable correlation with a standard signal ratio; and
(d) selecting at least one perfect match pre-optimized oligonucleotide, wherein the perfect match pre-optimized oligonucleotide has an acceptable relative standard deviation.

4. The method of claim 1, wherein said pre-optimized oligonucleotide is further defined as being selected by the steps of:
providing a sample comprising at least one target polynucleotide;
subjecting said sample to an array of oligonucleotides, wherein the hybridization of said target polynucleotide to at least one oligonucleotide in the array provides a detectable hybridization fingerprint; and
identifying at least one optimal oligonucleotide from said fingerprint.

5. The method of claim 1, wherein said pre-optimized oligonucleotide is further defined as being selected by the steps of:
providing a sample comprising a plurality of target polynucleotides, said target polynucleotides defined as RNA polynucleotides from more than one gene;
subjecting said sample to an array of oligonucleotides, wherein the hybridization of more than one different RNA polynucleotide to a respective oligonucleotide in the array provides a detectable hybridization fingerprint for more than one gene; and
identifying at least one optimal oligonucleotide for said more than one gene from said fingerprint.

6. The method of claim 1, wherein said identifying step utilizes an algorithm to identify said oligonucleotide.

7. The method of claim 6, wherein said algorithm identifies an oligonucleotide having complete complementarity to at least a portion of said target polynucleotide.

8. The method of claim 1, wherein the target polynucleotide is comprised in a plurality of RNA polynucleotides and the concentration of said plurality is from about 1 μg to about 10 μg.

9. The method of claim 1, wherein said ligand comprises an antibody.

10. The method of claim 1, wherein the label of the target polynucleotide and/or the label of the ligand comprises a fluorescent label, an enzyme label, a chemical label, or a gold label.

11. The method of claim 1, wherein the label of the target polynucleotide and the label of the ligand are identical.

12. The method of claim 1, wherein said microsphere is comprised in a plurality of microspheres and said target polynucleotide is comprised in a plurality of RNA polynucleotides.

13. The method of claim 12, wherein the plurality of RNA polynucleotides is comprised in a mRNA-containing sample, and said method is further defined as a method for providing mRNA expression profiling information.

14. The method of claim 12, wherein at least one microsphere in said plurality of microspheres comprises different oligonucleotides from the oligonucleotides of another microsphere in said plurality.

15. The method of claim 12, wherein at least one microsphere in the plurality comprises more than one non-identical pre-optimized oligonucleotide having sequence complementary to the same RNA polynucleotide.

16. A composition, comprising:
a plurality of microspheres, each microsphere linked to at least one pre-optimized oligonucleotide, wherein said oligonucleotide is hybridized to a labeled RNA polynucleotide forming an oligonucleotide/labeled RNA polynucleotide hybridized complex, and wherein said complex comprises a detectable signal through the binding of a receptor to the label, said signal amplified upon binding of a labeled ligand for the receptor.

17. The composition of claim 16, wherein at least one microsphere in said plurality of microspheres comprises different oligonucleotides from the oligonucleotides of another microsphere in said plurality.

18. The composition of claim 16, wherein at least one microsphere in the plurality comprises more than one non-identical pre-optimized oligonucleotide each having sequence complementary to the same RNA polynucleotide.

19. A method of optimizing an oligonucleotide hybridization-based assay, comprising the steps of:
providing a sample comprising at least one target polynucleotide;
subjecting said sample to an array of oligonucleotides, wherein the hybridization of said target polynucleotide to at least one oligonucleotide in the array provides a detectable hybridization fingerprint;
identifying at least one optimal oligonucleotide from said fingerprint, wherein said identifying step utilizes an algorithm defined by at least one of the following selection criteria:
(a) selecting at least one perfect match pre-optimized oligonucleotide, wherein the selected at least one perfect match pre-optimized oligonucleotide has an acceptable measure of correlation with a standard gene expression value;
(b) selecting at least one perfect match and minus mismatch pre-optimized oligonucleotide pair, wherein within a pair the selected at least one perfect match pre-optimized oligonucleotide minus the mismatch pre-optimized oligonucleotide has an acceptable measure of correlation with a standard gene expression value;
(c) selecting at least one pair of pre-optimized oligonucleotides from different pre-optimized oligonucleotide sets, wherein the ratio of signals in the pre-optimized oligonucleotides in the at least one pair of pre-optimized oligonucleotides has an acceptable correlation with a standard signal ratio; and
(d) selecting at least one perfect match pre-optimized oligonucleotide, wherein the perfect match pre-optimized oligonucleotide has an acceptable relative standard deviation; and
subjecting said optimal oligonucleotide to an oligonucleotide hybridization-based assay.
